SEQUENCE LISTING (4 sequences)

SEQ ID NO: 1 - DNA, 21 bp, Artificial Sequence
  F Primer
tgtgcctctgtgtctcgttta

SEQ ID NO: 2 - DNA, 20 bp, Artificial Sequence
  R Primer
accaccatcccctgatttgg

SEQ ID NO: 3 - DNA, 20 bp, Artificial Sequence
  F Primer
agagctacgagctgcctgac

SEQ ID NO: 4 - DNA, 20 bp, Artificial Sequence
  R Primer
agcactgtgttggcgtacag

(19) United States
(12) Patent Application Publication
(10) Pub. No.: US 2018/0208653 A1
(43) Pub. Date: Jul. 26, 2018

(54) METHODS FOR ENHANCING AN IMMUNE RESPONSE

(71) Applicant: Beth Israel Deaconess Medical Center, Boston, MA (US)

(72) Inventor: George C. TSOKOS, Brookline, MA (US)

(21) Appl. No.: 15/875,695

(22) Filed: Jan. 19, 2018

Related U.S. Application Data
(60) Provisional application No. 62/448,453, filed on Jan. 20, 2017.

(51) Int. Cl. C07K 16/28; A61P 37/04; A61K 39/39

(52) U.S. Cl. CPC C07K 16/28; A61K 2039/505; A61K 39/39; A61P 37/04

(57) ABSTRACT

Methods and compositions for enhancing an immune response in a subject (e.g., an immunocompromised subject or a subject with systemic lupus erythematosus (SLE)) are described. In particular, methods of administering to the subject an effective amount of an anti-signaling lymphocytic activation molecule family member 7 (SLAMF7)-antibody or antigen binding fragment thereof are described.

### FIELD OF THE INVENTION

The present invention relates to methods and compositions for enhancing an immune response in a subject with systemic lupus erythematosus (SLE). In particular, embodiments of the invention relate to treating or preventing an infectious disease in the subject with SLE by administering to the subject an anti-SLAMF antibody to treat or prevent the infectious disease in the subject.

### CROSS-REFERENCE TO RELATED APPLICATION

This application claims priority to U.S. Provisional Patent Application No. 62/448,453, filed on Jan. 20, 2017, the disclosure of which is incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “689200.1U1 Sequence Listing” and a creating date of Jan. 19, 2018, and having a size of 1 kb. The sequence listing submitted via EFS-Web is part of the specification and is herein incorporated by reference in its entirety.

### BACKGROUND OF THE INVENTION

Profound abnormalities characterize T cells from patients with Systemic Lupus Erythematosus (SLE) [1, 2]. Even though CD4+ T cells have been extensively studied, the role of CD8+ T cells in SLE is less well understood. Studies suggest a defect in cytotoxic CD8+ T cell activity in SLE and an accelerated disease in lupus prone mice in which the major killing pathways of cytotoxic CD8+ T cells have been deleted [3-5]. Additionally, SLE has been associated with defective cytotoxic responses against foreign antigens, an abnormality considered to contribute to the increased rate of infections, the leading cause of mortality in SLE [6-8].

Signaling lymphocytic activation molecule (SLAM) family members are type I transmembrane glycoprotein cell surface receptors that deliver downstream signals upon their engagement and modulate the magnitude of the immune response [9]. SLAM family member 7 (SLAMF7; CS1, CRACC, CD319) is expressed on natural killer (NK) cells, NK T cells, CD8+ T cells, and subsets of B cells [10]. SLAMF7 acts as a self-ligand through the binding of the N-terminal Ig domain of SLAMF7 [10]. In NK cells, SLAMF7 uses the adaptor protein Ewing's sarcoma's/FLI1-activated transcript 2 (EAT-2) to promote downstream signaling, while other SLAMF molecules preferentially bind to SLAMF associated protein (SAP) [11].

The role of SLAMF7 has been extensively studied in patients with multiple myeloma, a malignant disorder of plasma cells. A monoclonal antibody (mAb) directed against SLAMF7 has been shown to eliminate myeloma cells by activating NK cell and by stimulating antibody-dependent cellular cytotoxicity [12-14]. SLAMF molecules, and their downstream adaptor protein SAP, have been suggested to play a role in SLE pathogenesis and to represent a potential therapeutic target [15-17]. However, limited data are available concerning the role of SLAMF7 in SLE and some studies have proposed that SLAMF7 can play a role in the pathogenesis of the disease [18, 19]. The gene encoding SLAMF7 is located on chromosome 1 within the 1q23 locus, a region known to be associated with an increased susceptibility for SLE [20]. Moreover, SLAMF7 expression has been reported to be altered in NK cells, plasmacytoid dendritic cells, and a subset of B cells isolated from the peripheral blood of SLE patients [18, 19], but the role and function of SLAMF7 in SLE CD8+ T cells has not been established.

As described herein, the frequency of effector CD8+ T cells was decreased in the peripheral blood of SLE patients compared to healthy controls. Additionally, it was shown that the expression of SLAMF7 was decreased in SLE CD8+ T cells and that the presence of SLAMF7 characterized cytotoxic CD8+ T cells. Further, engagement of SLAMF7 with a specific monoclonal antibody (mAb) enhanced degranulation and cytotoxicity of CD8+ T cells in response to viral peptides.

Accordingly, despite the reduced SLAMF7 expression on SLE CD8+ effector T cells, SLAMF7 engagement via an anti-SLAMF7 mAb restored the defective SLE effector CD8+ T cell function in response to viral antigens and therefore represents a promising therapeutic target to enhance immune response against foreign antigen.

### BRIEF SUMMARY OF THE INVENTION

The present invention relates to methods of enhancing an immune response in an immunocompromised subject (e.g., a subject with systemic lupus erythematosus (SLE)). Subjects with SLE are generally provided immunosuppressive drugs, which can accentuate the rate of infection in the subject given that SLE is an autoimmune disease. Thus, provided herein are methods of enhancing an immune response in a subject. The methods of enhancing an immune response can, for example, comprise treating or preventing infectious diseases in the immunocompromised subjects.

In one general aspect, provided herein are methods of enhancing an immune response in a subject with systemic lupus erythematosus (SLE). The methods comprise administering to the subject an effective amount of an anti-signaling lymphocytic activation molecule family member (SLAMF) antibody (e.g., SLAMF7) or antigen binding fragment thereof, wherein administration of an effective amount of the anti-SLAMF antibody (e.g., SLAMF7) or antigen binding fragment thereof results in an enhanced immune response in the subject with SLE.

In another general aspect, provided herein are methods of enhancing an immune response in an immunocompromised subject. The methods comprise administering to the immunocompromised subject an effective amount of an anti-signaling lymphocytic activation molecule family member (SLAMF)-antibody (e.g., SLAMF7) or antigen binding fragment thereof. Administration of an effective amount of the anti-SLAMF antibody (e.g., SLAMF7) or antigen binding fragment thereof results in an enhanced immune response in the immunocompromised subject.

In certain embodiments, the anti-SLAMF antibody or antigen binding fragment thereof is selected from the group consisting of a synthetic antibody, a multi-specific antibody, a chimeric antibody, a single domain antibody, a heavy chain-only antibody, a polyclonal antibody, and a monoclonal antibody. In certain embodiments, the anti-SLAMF antibody is a monoclonal antibody. In certain embodiments, the SLAMF antibody or antigen binding fragment thereof is a humanized antibody or humanized antigen binding fragment thereof.

In certain embodiments, enhancing an immune response comprises treating or preventing an infectious disease in the subject (e.g., the immunocompromised subject, the subject with SLE). The infectious disease can, for example, be selected from the group consisting of a bacterial infection, a viral infection, and a fungal infection.

In certain embodiments, the anti-SLAMF antibody (e.g., SLAMF7) or antigen binding fragment thereof is administered to the subject in a pharmaceutical composition. The pharmaceutical composition can further comprise an adjuvant. The pharmaceutical composition can, for example, be administered prophylactically or therapeutically. In certain embodiments, the route of administration of the pharmaceutical composition to the subject is selected from the group consisting of intramuscularly, subcutaneously, parenterally, orally, intranasally, intradermally, transdermally, intraperitoneally, intratracheally, topically, rectally, and pulmonarily.

Also provided herein are methods of treating or preventing an infectious disease in a subject with systemic lupus erythematosus (SLE). The methods comprise administering to the subject an effective amount of an anti-signaling lymphocytic activation molecule family member (SLAMF)-antibody (e.g., SLAMF7) or antigen-binding fragment thereof, wherein administration of an effective amount of the anti-SLAMF antibody (e.g., SLAMF7) or antigen binding fragment thereof results in the treatment or prevention of the infectious disease in the subject with SLE.

In certain embodiments, the infectious disease is selected from the group consisting of a bacterial infection, a viral infection, and a fungal infection.

In certain embodiments, the SLAMF is selected from the group consisting of SLAMF1, SLAMF2, SLAMF3, SLAMF4, SLAMF5, SLAMF6, SLAMF7, SLAMF8, and SLAMF9. In certain embodiments, the SLAMF is SLAMF7.

Also provided are methods of enhancing degranulation and cytotoxicity of CD8+ T cells in a subject in need of treatment of systemic lupus erythematosus (SLE). The methods comprise administering to the subject an effective amount of an antibody that binds a signaling lymphocytic activation molecule family member (SLAMF) (e.g., SLAMF7), or an antigen binding fragment thereof, wherein administration of the antibody that binds SLAMF (e.g., SLAMF7) or antigen binding fragment thereof to the subject results in enhanced degranulation and cytotoxicity of CD8+ T cells in the subject.

Also provided are methods of enhancing degranulation and cytotoxicity of CD8+ T cells in an immunocompromised subject. The methods comprise administering to the subject an effective amount of an anti-signaling lymphocytic activation molecule family member (SLAMF)-antibody (e.g., SLAMF7) or antigen binding fragment thereof. Administration of the anti-SLAMF-antibody (e.g., SLAMF7) or antigen binding fragment thereof to the subject results in an enhancement of degranulation and cytotoxicity of CD8+ T cells in the immunocompromised subject.

Also provided are methods of treating systemic lupus erythematosus (SLE) in a subject. The methods comprise administering to the subject an effective amount of an anti-signaling lymphocytic activation molecule family member (SLAMF)-antibody (e.g., SLAMF7) or antigen binding fragment thereof, wherein administration of the anti-SLAMF-antibody (e.g., SLAMF7) or antigen binding fragment thereof to the subject results in the treatment of SLE in the subject.

### BRIEF DESCRIPTION OF THE DRAWINGS

The foregoing summary, as well as the following detailed description of the invention, will be better understood when read in conjunction with the appended drawings. For the purpose of illustrating the invention there are shown in the drawings embodiments which are presently preferred. It should be understood, however, that the invention is not limited to the precise arrangements and instrumentalities shown. In the drawings:

FIGS. 1A-1F show the distribution of CD8+ T cell differentiated subsets in peripheral blood from SLE patients. FIG. 1A shows a histogram of PBMC isolated from SLE patients that were stained for CD8+ T cell differentiated subsets by examining the expression of CCR7 and CD45RA. FIG. 1B shows a graph of the distribution of CD8+ T cell differentiated subsets in SLE patients compared to healthy controls. FIGS. 1C-1F show graphs of the frequency of naive CD8+ T cells (FIG. 1C); CM (FIG. 1D); EM (FIG. 1E); and terminally differentiated effector memory (TDEM) CD8+ T cells (FIG. 1F) in three groups of cohorts: inactive SLE (SLEDAI<4), active SLE (SLEDAI≥4) and healthy control (CON). Naïve (CCR7+CD45RA+); CM: Central Memory (CCR7+CD45RA−); EM: Effector Memory (CCR7−CD45RA−); TDEM: Terminally Differentiated Effector Memory (CCR7−CD45RA+); DN: double negative (CD3+CD4−CD8−) (mean±SEM; SLE n=44, controls n=41).

FIG. 2 shows a graph of the distribution of CD8+ T cell differentiated subsets in peripheral blood from SLE patients. The frequency of total CD8 in the peripheral blood of SLE patients is shown in the graph in the left panel. The CD8+ T cell distribution in inactive SLE (SLEDAI<4), active SLE (SLEDAI≥4), and control (CON) is shown in the graph on the right panel. (mean±SEM; SLE n=44, controls n=41).

FIGS. 3A-3G show that SLAMF7 expression is reduced on CD8+ T cells isolated from SLE patients compared to healthy controls. SLAMF7 expression was assessed by flow cytometry on T Cells isolated from peripheral blood. FIG. 3A shows a graph of the frequency (%) of SLAMF7 expression on CD4+, CD8+, and double negative (DN) T cells isolated from SLE patients and controls. FIG. 3B shows a graph demonstrating expression frequency (SLAMF7 expression as % of total CD8+ cells) and correlation with disease activity (inactive SLE (SLEDAI<4); active SLE (SLEDAI≥4), and healthy control (CON)). FIGS. 3C and 3D show histograms demonstrating the frequency of SLAMF7 expression on central memory (CM), effector memory (EM), and terminally differentiated effector memory (TDEM) CD8+ cells. FIGS. 3E-3G show graphs of the assessment of SLAMF7 expression by CM (FIG. 3E), EM (FIG. 3F), and TDEM (FIG. 3G) CD8+ T differentiated subsets in three cohorts: inactive SLE (SLEDAI<4); active SLE (SLEDAI≥4), and healthy control (CON) (mean±SEM; SLE n=16 to 27, controls n=13 to 22).

FIG. 4 shows that EAT-2 is not expressed by SLAMF7− and SLAMF7+ CD8+ T cells. FIG. 4 shows a graph demonstrating expression of EAT-2 as assessed by quantitative PCR (qRT-PCR) in sorted NK cells, CD8+ SLAMF7− cells and CD8+ SLAMF7+ cells (mean±SEM; n=3).

FIGS. 5A-5B show the expression of perforin, GzmA, and GzmB is restricted to the SLAMF7+ CD8+ T cell population. The frequency of CD8+ T cells expressing perforin, GzmA, and GzmB was assessed by flow cytometry. FIG. 5A shows representative flow cytometric profiles of SLAMF7 expression vs perforin, granzyme A, and granzyme B expression. The numbers indicate percentages. FIG. 5B shows a graph of cumulative data of % of total CD8+ T cells vs perforin, GzmA, and GzmaB expression in SLE patients and healthy controls (mean±SEM, SLE n=18, controls n=15).

FIGS. 6A-6B show that SLAMF7 engagement restores effector function of SLE CD8+ T cells in response to antigenic stimulation. PBMC from SLE and controls were stimulated with CEF (CMV-EBV-Flu peptide mix) for 6 hours in the presence of an anti-SLAMF7 monoclonal antibody. CD107a expression and IFNγ production were assessed by flow cytometry (FIG. 6A) at the end of the stimulation. SEB (Staphylococcal Enterotoxin B) was used as a positive control. FIG. 6B shows a graph of IFNγ+CD 107a expression in as % of total CD8+ T cells in unstimulated cells, CEF stimulated cells, and CEF stimulated cells treated with anti-SLAMF7 monoclonal antibody.

FIGS. 7A-7C show that SLAMF7 enhances cytotoxic activity of CD8+ T cells in response to viral antigens. CEF stimulated PBMC were enriched for CD8+ T cells after 6 days of stimulation. Subsequently, CD8+ T cells were treated with an anti-SLAMF7 monoclonal antibody (or an isotype control) and co-cultured for 6 hours with autologous CEF pulsed CD4+ T cells and un-pulsed CD4+ T cells. CEF specific killing of target CD4+ T cells was expressed as aqua positive cells in response to different ratios of CD8+ T cells. Un-pulsed CD4+ T cells were used as a control to determine background target cell death. FIG. 7A shows representative dot plots showing CD4+ T cells killing in response to SLAMF7 or isotype control treated CD8+ T cells. FIGS. 7B and 7C show graphs of the percentage of CEF-specific target cell lysis in PBMCs from healthy controls (FIG. 7B) and SLE patients (FIG. 7C). Results are expressed as [Aqua+ pulsed CD4+ T cells]−[Aqua+ un-pulsed CD4+ T cells]. Bars show the mean±SEM; n=4 controls and 5 SLE patients). ***=P<0.001.

FIG. 8 shows the gating strategy for the cytotoxic assay. Live and dead cells were gated from FSC-A/SSC-A dot plot. From live and dead cells, CD4+ T cells were analyzed for CFSE expression, allowing distinction between CEF peptide loaded CD4+ T cells (CFSE high) and un-pulsed CD4+ T cells (CFSE low).

### DETAILED DESCRIPTION OF THE INVENTION

Various publications are cited or described in the background and throughout the specification and each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. It must be noted that as used herein and in the appended claims, the singular forms “a,” “an,” and “the” include plural reference unless the context clearly dictates otherwise.

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Provided herein are methods of enhancing an immune response in a subject with systemic lupus erythematosus (SLE). The methods can comprise administering to the subject an effective amount of an anti-signaling lymphocytic activation molecule family member (SLAMF)-antibody (e.g., SLAMF7) or antigen binding fragment thereof. Administration of an effective amount of the anti-SLAMF antibody (e.g., SLAMF7) or antigen binding fragment thereof results in an enhanced immune response in the subject with SLE.

Also provided herein are methods of enhancing an immune response in an immunocompromised subject. The methods can comprise administering to the immunocompromised subject an effective amount of an anti-signaling lymphocytic activation molecule family member (SLAMF)-antibody (e.g., SLAMF7) or antigen binding fragment thereof. Administration of an effective amount of the anti-SLAMF antibody (e.g., SLAMF7) or antigen binding fragment thereof results in an enhanced immune response in the immunocompromised subject.

In certain embodiments, enhancing an immune response in the subject can comprise treating or preventing an infectious disease in the subject with SLE. The infectious disease can, for example, be selected from the group consisting of a bacterial infection, a viral infection, and a fungal infection.

Also provided are methods of enhancing degranulation and cytotoxicity of CD8+ T cells in a subject in need of treatment of systemic lupus erythematosus (SLE). The methods comprise administering to the subject an effective amount of an antibody that binds a SLAMF (e.g., a SLAMF7), or an antigen binding fragment thereof. Administration of the anti-SLAMF antibody (e.g., SLAMF7) or antigen binding fragment thereof to the subject results in enhancing degranulation and cytotoxicity of CD8+ T cells in the subject in need of treatment of SLE.

Also provided are methods of enhancing degranulation and cytotoxicity of CD8+ T cells in an immunocompromised subject. The methods comprise administering to the subject an effective amount of an anti-signaling lymphocytic activation molecule family member (SLAMF)-antibody (e.g., SLAMF7) or antigen binding fragment thereof. Administration of the anti-SLAMF-antibody (e.g., SLAMF7) or antigen binding fragment thereof to the subject results in an enhancement of degranulation and cytotoxicity of CD8+ T cells in the immunocompromised subject.

Degranulation and cytotoxicity of CD8+ T cells in response to antigens (e.g., viral, bacterial, or fungal antigens) in a subject (e.g., a subject with SLE or an immunocompromised subject) can be restored by binding of an anti-SLAMF7-antibody to SLAMF7. Degranulation of CD8+ T Cells in response to engagement of SLAMF7 with the anti-SLAMF7-antibody can, for example, be determined by examining expression levels of CD3, CD8, CD107a, and interferon γ (IFNγ). Enhancing degranulation and cytotoxicity of the CD8+ T cells in the subject by administration of the anti-SLAMF7 antibody or antigen binding fragment thereof can result in an enhanced immune response when the subject is exposed to an exogenous antigen. Administration of the anti-SLAM7 antibody to the subject (e.g., a subject with SLE or an immunocompromised subject) can, for example, restore the immune function in the subject (e.g., a subject with SLE or an immunocompromised subject) to that of a healthy subject.

Also provided herein are methods of treating systemic lupus erythematosus (SLE) in a subject. The methods can, for example, comprise administering to the subject an effective amount of an anti-signaling lymphocytic activation molecule family member (SLAMF)-antibody (e.g., SLAMF7) or antigen binding fragment thereof. Administration of the anti-SLAMF-antibody or antigen binding fragment thereof to the subject results in the treatment of SLE in the subject.

As used herein, the term “subject” refers to an animal, preferably a mammal, and may include a non-primate (e.g., a pig, horse, goat, sheep, cat, dog, rabbit, rat, or mouse) and a primate (e.g., a monkey, chimpanzee, or a human). In certain embodiments, a subject is a non-human animal. In another embodiment, a subject is a human. The terms “subject” and “patient” can be used herein interchangeably. The compositions according to the invention are suitable for administration to a subject, and can be used to treat or prevent an infectious disease in a subject with systemic lupus erythematosus (SLE).

As used herein, the term “treating,” “treat,” and/or “treatment” refers to partially or completely inhibiting or reducing the condition (e.g., systemic lupus erythematosus (SLE) or infectious disease) from which the subject is suffering. In one embodiment, the term refers to an action that occurs while a patient is suffering from, or is diagnosed with, the condition, which reduces the severity of the condition, or slows the progression of the condition. Treatment need not result in a complete cure of the condition; partial inhibition or reduction of the condition in the subject is encompassed by this term. Treatment is intended to encompass prevention or prophylaxis.

As used herein, the term “prevent,” “preventing,” and/or “prevention” refers to an action that occurs before the subject begins to suffer from the condition (e.g., systemic lupus erythematosus (SLE) or infectious disease), or relapse of the condition. The prevention need not result in a complete prevention of the condition; partial prevention or reduction of the condition to be treated, or reduction of the risk of the condition to be treated, is encompassed by this term.

As used herein, an “effective amount” refers to a minimal amount or concentration of an anti-SLAMF antibody or antigen binding fragment thereof (e.g., an anti-SLAMF7 antibody or antigen binding fragment thereof) that, when administered alone or in combination, is sufficient to provide a therapeutic benefit in the treatment or prevention of the condition (e.g., SLE or infectious disease), or to delay or minimize one or more symptoms associated with the condition. The term “therapeutically effective amount” can encompass an amount that improves overall therapy, reduces or avoids symptoms or causes of the condition, or enhances the therapeutic efficacy of another therapeutic agent. The therapeutic amount need not result in a complete cure of the condition; partial inhibition or reduction of the condition is encompassed by this term. The therapeutically effective amount can also encompass a prophylactically effective amount.

As used herein, “immunocompromised subject” refers to a subject with an impaired or weakened immune system. An immunocompromised subject can include, but is not limited, a subject with AIDS, a subject with cancer, a subject undergoing a transplant, a subject taking immunosuppressive drugs, a subject with a primary immunodeficiency disorder, and a subject with an autoimmune disease. Examples of autoimmune diseases can include, but are not limited to, congenital agammaglobulinemia, congenital IgA deficiency, rheumatoid arthritis, lupus, celiac disease, Sjogren's syndrome, polymyalgia rheumatic, multiple sclerosis, ankylosing spondylitis, alopecia areata, inflammatory bowel disease, type 1 diabetes, Guillain-Barre syndrome, psoriasis, Graves' disease, Hashimoto's thyroiditis, and vasculitis.

As used herein, “infectious disease” refers to a disease caused by a bacterial infection, a viral infection, a yeast infection, a mycoplasma infection, and/or a fungal infection, such that the infection can be spread by another organism to the subject at risk of obtaining the infectious disease. Infectious diseases can include, but are not limited to, anaplasmosis, anthrax, babesiosis, botulism, brucellosis, burkholderia mallei (glanders), burkholderia pseudomallei (melioidosis), campylobacteriosis, carbapenem-resistant enterobacteriaceae, chancroid, chikungunya, chlamydia, ciguatera, clostridium difficile infection, clostridium perfringens, coccidioidomycosis fungal infection, Creutzfeldt-Jacob disease, cryptosporidiosis, cyclosporiasis, dengue fever, diphtheria, E. coli infection, eastern equine encephalitis, ebola hemorrhagic fever (ebola), ehrlichiosis, encephalitis, enterovirus infection, giardiasis, gonococcal infection (gonorrhea), granuloma inguinale, haemophilus influenza disease, Type B (Hib or H-flu), hantavirus pulmonary syndrome, hemolytic uremic syndrome, hepatitis A (Hep A), hepatitis B (Hep B), hepatitis C (Hep C), hepatitis D (Hep D), hepatitis E (Hep E), herpes, herpes zoster (shingles), histoplasmosis infection, human immunodeficiency virus/AIDS (HIV/AIDS), human papillomarivus (HPV), influenza (flu), legionnaires disease, leprosy, leptospirosis, listeriosis, lyme disease, lymphogranuloma venereum infection, malaria, measles, meningococcal disease (bacterial, viral), middle east respiratory syndrome coronavirus (MERS-CoV), mumps, norovirus, paralytic shellfish poisoning, pediculosis (head and body lice), pelvic inflammatory disease, pertussis, plague (bubonic, septicemic, pneumonic), pneumococcal disease, poliomyelitis (polio), psittacosis, Q-fever, rabies, rickettsiosis (rocky mountain spotted fever), rubella, salmonellosis gastroenteritis (salmonella), scabies infestation, scombroid, severe acute respiratory syndrome (SARS), shigellosis gastroenteritis (shigella), smallpox, staphylococcal infection, streptococcal disease, streptococcal toxic-shock syndrome, syphilis, tetanus infection, trichonosis, tuberculosis, tularemia (rabbit fever), typhoid fever, typhus, vaginosis (yeast infection), varicella (chickenpox), vibrio cholera, vibriosis, viral hemorrhagic fever, west nile virus, yellow fever, yersenia, and zika virus.

As used herein, the term “SLAMF” refers to a family of genes referred to as signaling lymphocytic activation molecule family (SLAMF) members. SLAMF are type I transmembrane glycoprotein cell surface receptors that deliver downstream signals upon their engagement and modulate the magnitude of the immune response. Homophilic binding between SLAMF is involved in cellular adhesion. SLAMF are CD2-related surface receptors expressed by activated T cells and B cells. SLAMF members include SLAMF1 (CD150), SLAMF2 (CD48), SLAMF3 (CD229, LY9), SLAMF4 (CD244), SLAMF5 (CD84), SLAMF6 (CD352), SLAMF7 (CD319), CLAMF8 (CD353), and SLAMF9.

Provided herein are methods of enhancing an immune response in a subject (e.g., an immunocompromised subject or a subject with SLE). The methods comprise administering to the subject an anti-SLAMF antibody (e.g., SLAMF7) or antigen binding fragment thereof to the subject. In certain embodiments, the anti-SLAMF antibody or antigen binding fragment thereof is selected from the group consisting of a synthetic antibody, a multi-specific antibody, a chimeric antibody, a single domain antibody, a heavy chain-only antibody, a polyclonal antibody, and a monoclonal antibody. In certain embodiments, the anti-SLAMF antibody (e.g., SLAMF7) or antigen binding fragment thereof is a humanized antibody or humanized antigen binding fragment thereof.

### Antibodies

One of ordinary skill in the art will be familiar with the structure of an antibody. The light and heavy chains each contain a variable region that is responsible for binding the target antigen (e.g., SLAMF7). The variable region contains the antigen binding determinants of the molecule, thus determining the specificity of an antibody for its target antigen. The variable regions of the light and heavy chains each comprise three complementarity determining regions (CDRs).

As used herein “complementarity determining region” and “CDR” refer to an amino acid sequence of a variable region of a heavy or light chain of an antibody that contributes to specific recognition of, and binding specificity for, the antigen. The CDRs are referred to as CDR1, CDR2, and CDR3.

As used herein, “antibody or fragment thereof against SLAMF,” “antibody or fragment thereof that specifically binds SLAMF,” and “anti-SLAMF antibody or antigen binding fragment thereof,” shall all have the same meaning, and refer to an antibody or antigen binding fragment thereof, that binds specifically to a SLAMF, e.g., SLAMF7.

An “antibody fragment” or “antigen binding fragment” comprises a portion of an intact antibody, preferably the antigen binding and/or the variable region of the intact antibody. Examples of antibody fragments include, but are not limited to Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules; single-domain antibodies (such as V_{H}H), and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy-terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “V_{H}Hs” (Variable domain of the heavy chain of the Heavy chain antibody). Some V_{H}Hs may also be known as nanobodies. Camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). A basic V_{H}H has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

An “isolated” antibody (or construct) is one that has been identified, separated and/or recovered from a component of its production environment (e.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator; or (3) to homogeneity by SDS-PAGE under non-reducing or reducing conditions using Coomassie Blue or, preferably, silver stain. Isolated antibody (or construct) includes the antibody in situ within recombinant cells since at least one component of the antibody's natural environment will not be present. Ordinarily, however, an isolated polypeptide, antibody, or construct will be prepared by at least one purification step.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

As used herein a “chimeric antibody” is an antibody in which a portion of the heavy and/or light chain is identical with or homologous to corresponding sequences in an antibody derived from a particular species or belonging to a particular antibody class or subclass, while the remainder of the chain(s) is(are) identical with or homologous to corresponding sequences in an antibody derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). “Humanized antibody” is used as a subset of “chimeric antibodies.”

“Humanized” forms of non-human antibodies are chimeric antibodies that contain minimal sequence derived from non-human immunoglobulin. In some embodiments, a humanized antibody is a non-human immunoglobulin (recipient antibody), such as mouse, rat, rabbit, camel, llama, alpaca, or non-human primate, in which residues from a CDR of the recipient are replaced by residues from a CDR of a human species (donor antibody) having the desired specificity, affinity, and/or capacity. In some instances, framework (“FR”) residues of the non-human immunoglobulin are replaced by corresponding human residues. Furthermore, humanized antibodies may comprise residues that are not found in the recipient antibody or in the donor antibody. These modifications may be made to further refine antibody performance, such as binding affinity. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

As used herein, “binds specifically to” or “against” when used in connection with an antibody or fragment thereof and SLAMF7 refers to the binding or interaction between the antibody or fragment thereof and the SLAMF7. An antibody or fragment thereof according to the invention binds to SLAMF7 with a dissociation constant (K_{D}) of between 10⁻⁶ and 10⁻⁹ M, or less, and preferably with a dissociation constant of less than 10⁻⁹ M. The term “K_{D}” refers to the dissociation constant, which is obtained from the ratio of Kd to Ka (i.e., Kd/Ka) and is expressed as a molar concentration (M). K_{D} values for antibodies can be determined using methods in the art in view of the present disclosure. For example, the K_{D} of an antibody can be determined by using surface plasmon resonance, such as by using a biosensor system, e.g., a Biacore® system, or by using bio-layer interferometry technology, such as a Octet RED96 system.

The term “specificity” refers to selective recognition of an antigen binding protein, i.e., an antibody, for a particular epitope of an antigen. Natural antibodies, for example, are monospecific. The term “multispecific” as used herein denotes that an antigen binding protein has polyepitopic specificity (i.e., is capable of specifically binding to two, three, or more, different epitopes on one biological molecule or is capable of specifically binding to epitopes on two, three, or more, different biological molecules). “Bispecific” as used herein denotes that an antigen binding protein has two different antigen-binding specificities. The term “monospecific” as used herein denotes an antigen binding protein that has one or more binding sites each of which bind the same epitope of the same antigen.

Any method known in the art can be used for determining specific antigen-antibody binding including, for example, surface plasmon resonance (SPR), scatchard analysis and/or competitive binding assays, such as radioimmunoassay (RIA), enzyme immunoassays (EIA) and sandwich competition assays, and the different variants thereof known in the art, as well as other techniques mentioned herein. Methods for determining the binding affinities or dissociation constants are known to those skilled in the art.

Any method known in the art can be used for characterizing an antibody according to the invention, such as SDS-polyacrylamide gel electrophoresis (PAGE), circular dichroism (CD), size exclusion chromatography (SEC), etc. Methods for characterizing proteins, i.e. determining the oligomeric state, melting temperature, molecular weight, purity, etc., are known to those skilled in the art.

An antibody or fragment thereof according to embodiments of the invention can be produced recombinantly from a recombinant host cell using methods known in the art in view of the present disclosure. The recombinantly produced antibody or fragment thereof can be different from the naturally occurring antibody or fragment thereof, for example, in posttranslational modification of amino acids. As used herein, “posttranslational modification of amino acids” refers to any modification to the amino acids after translation of the amino acids, such as by attaching to one or more amino acids independently one or more biochemical functional groups (such as acetate, phosphate, various lipids and carbohydrates), changing the chemical nature of an amino acid (e.g. citrullination), or making structural changes (e.g. formation of disulfide bridges).

### Pharmaceutical Compositions

In certain embodiments, the anti-SLAMF antibody (e.g., SLAMF7) or antigen binding fragment thereof is administered to the subject in a pharmaceutical composition. Optionally, the pharmaceutical composition further comprises an adjuvant. The pharmaceutical composition can, for example, be administered prophylactically or therapeutically, wherein the route of administration of the pharmaceutical composition to the subject is selected from the group consisting of intramuscularly, subcutaneously, parenterally, orally, intranasally, intradermally, transdermally, intraperitoneally, intratracheally, topically, rectally, and pulmonarily.

The term “pharmaceutical formulation” of “pharmaceutical composition” refers to a preparation that is in such form as to permit the biological activity of the active ingredient (e.g., the anti-SLAMF antibody) to be effective, and that contains no additional components that are unacceptably toxic to a subject to which the formulation would be administered. Such formulations are sterile. A “sterile” formulation is aseptic or free from all living microorganisms and their spores.

A pharmaceutical composition can, for example, further comprises a pharmaceutically acceptable salt. The term “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

Pharmaceutical compositions described herein can further comprise a pharmaceutically acceptable carrier or pharmaceutically acceptable excipient. As used herein a “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the compositions.

The pharmaceutical compositions described herein may optionally additionally comprise a preservative, such as the mercury derivative thimerosal, phenoxyethanol, or parabens. In a specific embodiment, the pharmaceutical compositions described herein comprise 0.001% to 0.01% thimerosal. In other embodiments, the pharmaceutical compositions described herein do not comprise a preservative.

In certain embodiments, the pharmaceutical compositions described herein (e.g., the anti-SLAMF7 antibody or antigen binding fragment thereof) comprise, or are administered in combination with, an adjuvant. The adjuvant for administration in combination with a composition described herein can be administered before, concomitantly with, or after administration of said composition. In some embodiments, the term “adjuvant” refers to a compound that when administered in conjunction with or as part of a composition described herein augments, enhances and/or boosts the immune response to the antibody or the immune response to the antibody binding the antigen, but when the adjuvant compound is administered alone does not generate an immune response to the antibody or the antibody binding to the antigen. Adjuvants can enhance an immune response by several mechanisms including, e.g., lymphocyte recruitment, stimulation of B and/or T cells, and stimulation of macrophages. In certain embodiments, the compositions described herein do not comprise an adjuvant besides the antibodies and excipients, and/or are not administered in combination with an adjuvant besides the antibodies and the excipients.

Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see United Kingdom Patent GB2220211), MF59 (Novartis), AS03 (GlaxoSmithKline), AS04 (GlaxoSmithKline), imidazopyridine compounds (see WO2007/109812), imidazoquinoxaline compounds (see WO2007/109813) and saponins, such as QS21 (see Kensil et al., in Vaccine Design: The Subunit and Adjuvant Approach (eds. Powell & Newman, Plenum Press, N Y, 1995); U.S. Pat. No. 5,057,540). Other adjuvants are oil in water emulsions (such as squalene or peanut oil), optionally in combination with immune stimulants, such as monophosphoryl lipid A (see Stoute et al., 1997, N. Engl. J. Med. 336, 86-91). Another adjuvant is CpG (Bioworld Today, Nov. 15, 1998).

In certain embodiments, the compositions described herein are formulated to be suitable for the intended route of administration to a subject. For example, the compositions described herein can for instance be formulated to be suitable for intramuscular, subcutaneous, parenteral, oral, intranasal, intradermal, transdermal, colorectal, intraperitoneal, intratracheal, topical, rectal or pulmonary administration. In certain embodiments, the compositions described herein are useful for administration by intramuscular injection. In other embodiments, the compositions described herein can be administered intradermally. In other embodiments, the compositions described herein can be delivered via the skin.

### Embodiments

The invention provides also the following non-limiting embodiments.

Embodiment 1 is a method of enhancing an immune response in a subject with systemic lupus erythematosus (SLE), the method comprising administering to the subject an effective amount of an anti-signaling lymphocytic activation molecule family member 7 (SLAMF7)-antibody or antigen binding fragment thereof, wherein administration of an effective amount of the anti-SLAMF7 antibody or antigen binding fragment thereof results in an enhanced immune response in the subject with SLE.

Embodiment 2 is the method of embodiment 1, wherein the anti-SLAMF7 antibody or antigen binding fragment thereof is selected from the group consisting of a synthetic antibody, a multi-specific antibody, a chimeric antibody, a single domain antibody, a heavy chain-only antibody, a polyclonal antibody, and a monoclonal antibody.

Embodiment 3 is the method of embodiment 2, wherein the anti-SLAMF7 antibody or antigen binding fragment thereof is a monoclonal antibody.

Embodiment 4 is the method of embodiment 2 or 3, wherein the SLAMF7 antibody or antigen binding fragment thereof is a humanized antibody or a humanized antigen binding fragment thereof.

Embodiment 5 is the method of any one of embodiments 1-4, wherein enhancing an immune response comprises treating or preventing an infectious disease in the subject with SLE.

Embodiment 6 is the method of embodiment 5, wherein the infectious disease is selected from the group consisting of a bacterial infection, a viral infection, and a fungal infection.

Embodiment 7 is the method of any one of embodiments 1-6, wherein the anti-SLAMF7 antibody or antigen binding fragment thereof is administered to the subject in a pharmaceutical composition.

Embodiment 8 is the method of embodiment 7, wherein the pharmaceutical composition further comprises an adjuvant.

Embodiment 9 is the method of embodiment 7 or 8, wherein the pharmaceutical composition is administered prophylactically or therapeutically.

Embodiment 10 is the method of any one of embodiments 7-9, wherein the route of administration of the pharmaceutical composition to the subject is selected from the group consisting of intramuscularly, subcutaneously, parenterally, orally, intranasally, intradermally, transdermally, intraperitoneally, intratracheally, topically, rectally, and pulmonarily.

Embodiment 11 is a method of enhancing degranulation and cytotoxicity of CD8+ T cells in a subject in need of treatment of systemic lupus erythematosus (SLE), the method comprising administering to the subject an effective amount of an antibody that binds a signaling lymphocytic activation molecule family member 7 (SLAMF7), or an antigen binding fragment thereof, wherein administration of the antibody that binds SLAMF7, or an antigen binding fragment thereof to the subject results in an enhancement of degranulation and cytotoxicity of CD8+ T cells in the subject in need of treatment of SLE.

Embodiment 12 is the method of embodiment 11, wherein the anti-SLAMF7 antibody or antigen binding fragment thereof is selected from the group consisting of a synthetic antibody, a multi-specific antibody, a chimeric antibody, a single domain antibody, a heavy chain-only antibody, a polyclonal antibody, and a monoclonal antibody.

Embodiment 13 is the method of embodiment 12, wherein the anti-SLAMF7 antibody or antigen binding fragment thereof is a monoclonal antibody.

Embodiment 14 is the method of any one of embodiments 11-13, wherein the SLAMF7 antibody or antigen binding fragment thereof is a humanized antibody or humanized antigen binding fragment thereof.

Embodiment 15 is the method of any one of embodiments 11-14, wherein the anti-SLAMF7 antibody or antigen binding fragment thereof is administered to the subject in a pharmaceutical composition.

Embodiment 16 is the method of embodiment 15, wherein the pharmaceutical composition further comprises an adjuvant.

Embodiment 17 is the method of embodiment 15 or 16, wherein the pharmaceutical composition is administered prophylactically or therapeutically.

Embodiment 18 is the method of any one of embodiments 15-17, wherein the route of administration of the pharmaceutical composition to the subject is selected from the group consisting of intramuscularly, subcutaneously, parenterally, orally, intranasally, intradermally, transdermally, intraperitoneally, intratracheally, topically, rectally, and pulmonarily.

Embodiment 19 is a method of treating systemic lupus erythematosus (SLE) in a subject, the method comprising administering to the subject an effective amount of an anti-signaling lymphocytic activation molecule family member 7 (SLAMF7)-antibody or antigen binding fragment thereof, wherein administration of the anti-SLAMF7-antibody or antigen binding fragment thereof to the subject results in the treatment of SLE in the subject.

Embodiment 20 is a method of enhancing degranulation and cytotoxicity of CD8+ T cells in an immunocompromised subject, the method comprising administering to the subject an effective amount of an anti-signaling lymphocytic activation molecule family member 7 (SLAMF7)-antibody or antigen binding fragment thereof, wherein administration of the anti-SLAMF7-antibody or antigen binding fragment thereof to the subject results in an enhancement of degranulation and cytotoxicity of CD8+ T cells in the immunocompromised subject.

Embodiment 21 is a method of enhancing an immune response in an immunocompromised subject, the method comprising administering to the immunocompromised subject an effective amount of an anti-signaling lymphocytic activation molecule family member 7 (SLAMF7)-antibody or antigen binding fragment thereof, wherein administration of an effective amount of the anti-SLAMF7 antibody or antigen binding fragment thereof results in an enhanced immune response in the immunocompromised subject.

Embodiment 22 is the method of embodiment 21, wherein enhancing an immune response comprises treating or preventing an infectious disease in the immunocompromised subject.

Embodiment 23 is the method of embodiment 22, wherein the infectious disease is selected from the group consisting of a bacterial infection, a viral infection, and a fungal infection.

### EXAMPLES

Materials and Methods

Patients and Controls.

SLE patients (n=79) were diagnosed according to the American College of Rheumatology classification criteria [21], and were recruited from the Division of Rheumatology at Beth Israel Deaconess Medical Center. Age-, sex-, and ethnicity-matched healthy individuals were chosen as controls. Disease activity score was measured using the SLE Disease Activity Index (SLEDAI) [22] scoring system (Table 1). Informed consent was obtained as approved by the Institutional Review Board after the nature and possible consequences of the studies were explained.

TABLE 1
Characteristics of SLE patients

| Characteristics of SLE | |
|---|---|
| patients | (N = 79) |
| Age - yr | |
| Median | 41.3 |
| Range | 21-72 |
| Gender | |
| Female - (%) | 72 (91.1) |
| Male - (%) | 7 (8.9) |
| Ethnicity | |
| African American - (%) | 22 (27.8) |
| Asian - (%) | 5 (6.3) |
| Hispanic - (%) | 7 (8.9) |
| Caucasian - (%) | 40 (50.6) |
| Other - (%) | 5 (6.3) |
| SLE disease activity index | |
| (SLEDAI) | |
| Median | 3.6 |
| Range | (0-21) |
| Treatments | |
| Prednisone - (%) | 50 (63.3) |
| Hydroxychloroquine - (%) | 59 (74.7) |
| Mycophenolate Mofetil - (%) | 30 (38.0) |
| Azathioprine - (%) | 10 (12.7) |
| Methotrextate - (%) | 5 (6.3) |
| Belimumab - (%) | 2 (2.5) |
| Tacrolimus - (%) | 1 (1.3) |
| I.V. Immunoglobulins - (%) | 1 (1.3) |

Cell Isolation.

Peripheral blood mononuclear cells (PBMC) were enriched by density gradient centrifugation (Lymphocyte Separation Medium, Corning Life Sciences; Tewksbury, Mass., USA). T cells were isolated by negative selection (RosetteSep, Stem Cell Technologies; Vancouver, Calif.).

Flow Cytometry.

Cells were stained for dead cells (Zombie Aqua/NIR Fixable Viability Kit; Biolegend; San Diego, Calif.), and then labeled for surface antibodies (Table 2). Cells were permeabilized (Cytofix/Cytoperm, BD Biosciences; San Jose, Calif.) and stained for cytokines and/or cytotoxic enzymes. Data were acquired on a LSR II SORP (BD Biosciences) and analyzed using FlowJo (version 10.1r5, FlowJo Enterprise; Ashland, Oreg.).

TABLE 2
Antibodies

| | Format | Clone | Company |
|---|---|---|---|
| Flow cytometry | | | |
| Antibody | | | |
| anti-CD3 | BUV395 | SK7 | BD |
| anti-CD4 | APC | OKT4 | Biolegend |
| anti-CD4 | APC | OKT4 | Biolegend |
| anti-CD4 | PerCP eFLuor 710 | SK3 | eBioscience |
| anti-CD8 | PerCP | RPA-T8 | Biolegend |
| anti-CD8 | APC/Cy7 | RPA-T8 | Biolegend |
| anti-CD45RA | PE/Cy7 | HI100 | Biolegend |
| anti-CD45RA | APC/Cy7 | HI100 | Biolegend |
| anti-CD56 | APC | HCD56 | Biolegend |
| anti-CCR7 | Alexa Fluor 488 | G043H7 | Biolegend |
| anti-SLAMF7/CD319/ | PE | 162.1 | Biolegend |
| CS.1 | | | |
| anti-IFNγ | Pacific Blue | 4S.B3 | Biolegend |
| anti-CD107a (Lamp-1) | Pacific Blue | H4A3 | Biolegend |
| anti-Perforin | Pacific Blue | dG9 | Biolegend |
| anti-Granzyme A | Alexa Fluor 700 | CB9 | Biolegend |
| anti-Granzyme B | FITC | GB11 | Biolegend |
| mouse IgG2b k isotype | PE | MPC-11 | Biolegend |
| control | | | |
| Purified antibodies | | | |
| Antibody | | | |
| anti-CD3 | Purified | OKT3 | BioXcell |
| anti-CD28 | LEAF purified | 28.2 | Biolegend |
| anti-SLAMF7/CD319/ | Purified | 162.1 | Biolegend |
| CRACC | | | |
| mouse IgG2b k isotype | Purified | MPC-11 | Biolegend |
| control | | | EMD |
| Goat anti-mouse IgG | Puridied | Polyclonal | Millipore |
| crosslinker | | | |

Degranulation Assay.

Cryopreserved PBMC (2×10⁶) were stimulated for 6 hours in 1 ml of complete RPMI (RPMI 1640 supplemented with 10% fetal bovine serum, 100 mg/ml streptomycin, and 100 units/ml penicillin) in the presence of Golgiplug (1 μl/ml; BD; Franklin Lakes, N.J.) and Golgistop (1 μl/ml; BD), CD107a (4 μl/ml; Biolegend), and CEF (CMV-EBV-Flu) peptide mix (3 μg/ml) or Staphyloccal enterotoxin B (SEB; 1 μg/ml) as a positive control. When indicated, cells were also stimulated with anti-SLAMF7 mAb or a mouse IgG2b Isotype control antibody (0.5 g/ml, Biolegend), and a goat anti-mouse IgG cross-linker (EMD Millipore; Billerica, Mass.). At the end of the stimulation, cells were stained for live-dead cells (Zombie NIR Fixable Viability Kit, Biolegend), then stained for surface antibodies with CD3 (BD Horizon BUV395 anti-human CD3; BD), CD4 (PerCP eFluor 710 anti-human CD4; eBioscience; San Diego, Calif.), CD8 (PerCP anti-human CD8; Biolegend), fixed/permeabilized (Cytofix/cytoperm) and stained for IFNγ (Alexa Fluor 647 anti-human IFNγ; Biolegend). Cells were then acquired on an LSRII SORP and analyzed by using FlowJo (version 10.1r5, FlowJo Enterprise).

Flow Cytometry-Based Assay to Measure CD8+ T Cell Cytotoxic Activity in Response to Anti-SLAMF7 mAb Treatment.

Freshly isolated PBMCs were cultured in the presence of CEF peptide mix (5 μg/ml) and interleukin-2 (IL-2) (20 IU/ml; Peprotech; Rocky Hill, N.J.) for 6 days in complete RPMI. On day 3, half of the cell culture medium was replaced with fresh complete RPMI. On day 6, effector CD8+ T cells were isolated by negative selection using magnetic sorting (human CD8+ T cells isolation kit, Miltenyi Biotec; Bergisch Gladbach, Germany). Cells were re-suspended at a concentration of 10⁶/ml. Six (6) serial dilutions were performed. From each dilution, half of the cells were treated with anti-SLAMF7 mAb (5 μg/ml; Biolegend) and a goat anti-mouse IgG cross-linker (EMD Millipore), while the other half was treated with an IgG isotype control (Biolegend). 100 μl of each CD8+ T cell preparation were seeded in duplicate in a 96-well U bottom plate.

Freshly isolated autologous CD4+ T (human CD4+ T cells isolation kit, Miltenyi Biotec) were used as target. Half of the CD4+ T cells were labeled with 5,6-carboxyfluorescein succinimidyl ester (CFSE) 0.2 μM (CFSE high) and were pulsed with CEF peptide for 45 min at 37° C. in complete RPMI. The other half of the CD4+ T cells were labelled with CFSE 0.02 μM and were used as control to determine nonspecific background cell death (CFSE low). CFSE high and low were mixed at 1:1 ratio, and resuspended at a concentration of 2×10⁵ cells/ml. 100 μl of target CD4+ T cells were added to each dilution of CD8+ T cells. Cell mixtures were incubated for 6 hours (h) at 37° C.

After incubation, cells were stained for live and dead cells (Zombie Aqua Fixable Viability Kit; Biolegend), CD4 (APC anti-human CD4 Ab; Biolegend) and CD8 (APC/Cy7 anti-human CD8 Ab; Biolegend) and analyzed by Flow Cytometry. Aqua-positive staining indicated dead cells. The percentage of target cell lysis in response to effector CD8+ T cells was expressed as [% of Aqua+ pulsed CD4+ T cells]−[% Aqua+ unpulsed CD4+ T cells].

Real-Time Quantitative Reverse Transcriptase-Polymerase Chain Reaction (Q-RT-PCR).

Total RNA was extracted by RNeasy Mini Kit (Qiagen; Hilden, Germany). Reverse transcription was performed from 500 ng total RNA using the High Capacity cDNA Archive Kit (Applied Biosystems; Foster City, Calif.). Quantitative (Q)-RT-PCR was performed with 40 cycles at 94° C. (for 12 seconds) and 60° C. (for 60 seconds) using SYBR Green (LightCycler 4800 SYBR Green I Master, Roche; Basel Switzerland). The comparative Ct method was used to quantify transcripts relative to the endogenous control gene β-actin. Primer sequences were as follows: EAT-2, forward 5′-TGTGCCTCTGTGTCTCGTTTA-3′ (SEQ ID NO:1) and reverse 5′-ACCACCATCCCCTGATTTGG-3′ (SEQ ID NO:2); β-actin, forward 5′-AGAGCTACGAGCTGCCTGAC-3′ (SEQ ID NO:3) and reverse 5′-AGCACTGTGTTGGCGTACAG-3′ (SEQ ID NO:4).

Statistical Analysis.

Statistical analysis was performed using Student's t-test, corrected with the Holm-Sidak method. For multiple comparisons, statistical analysis was performed using one-way analysis of variance (Anova), followed by post hoc analysis with Tukey's test. Statistical analyses and illustrations were performed using FlowJo (version 10.1r5, FlowJo Enterprise), and GraphPad Prism (version 6).

### Results

### Example 1: Skewed Distribution of CD8+ T Cell Subsets in Peripheral Blood of SLE Patients

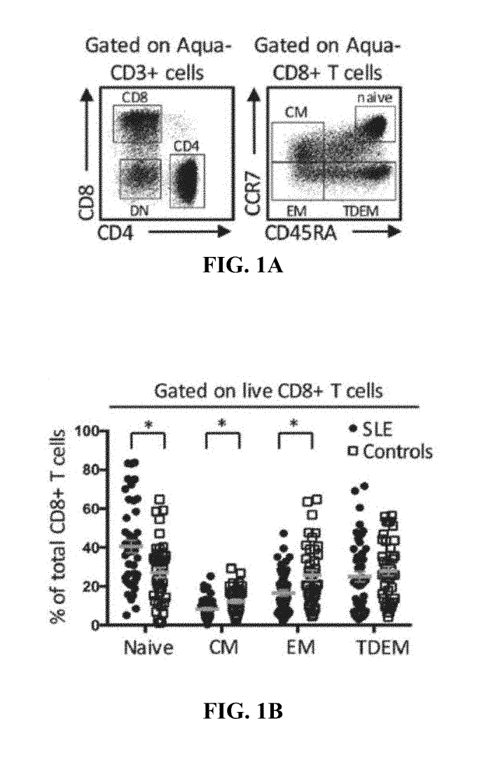

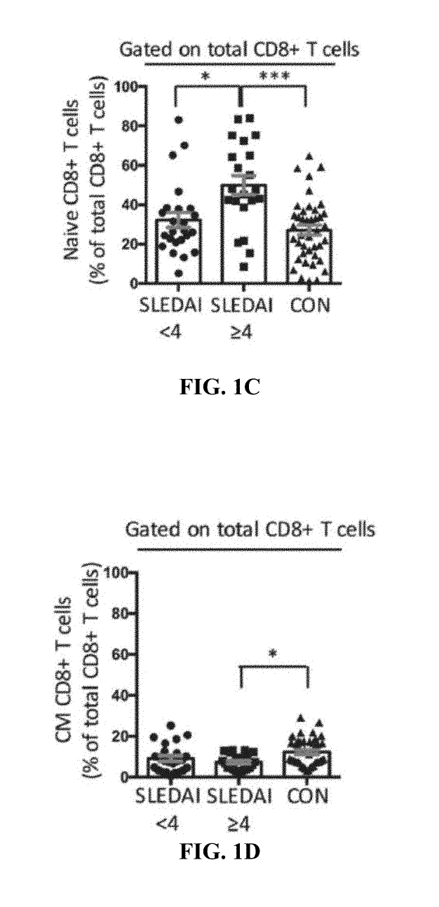

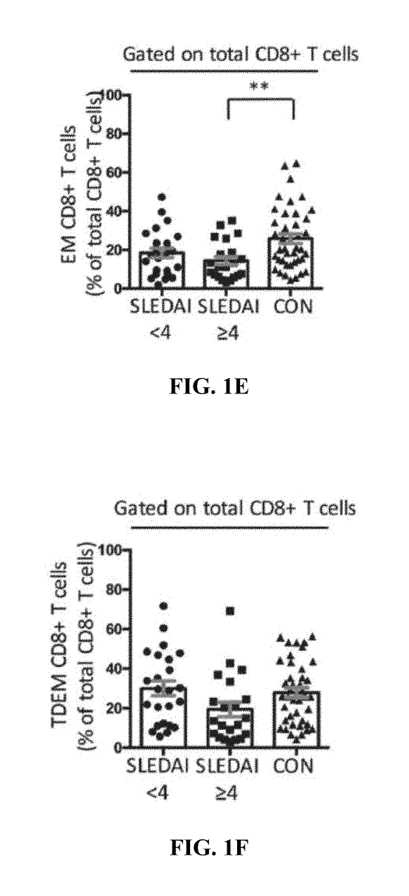

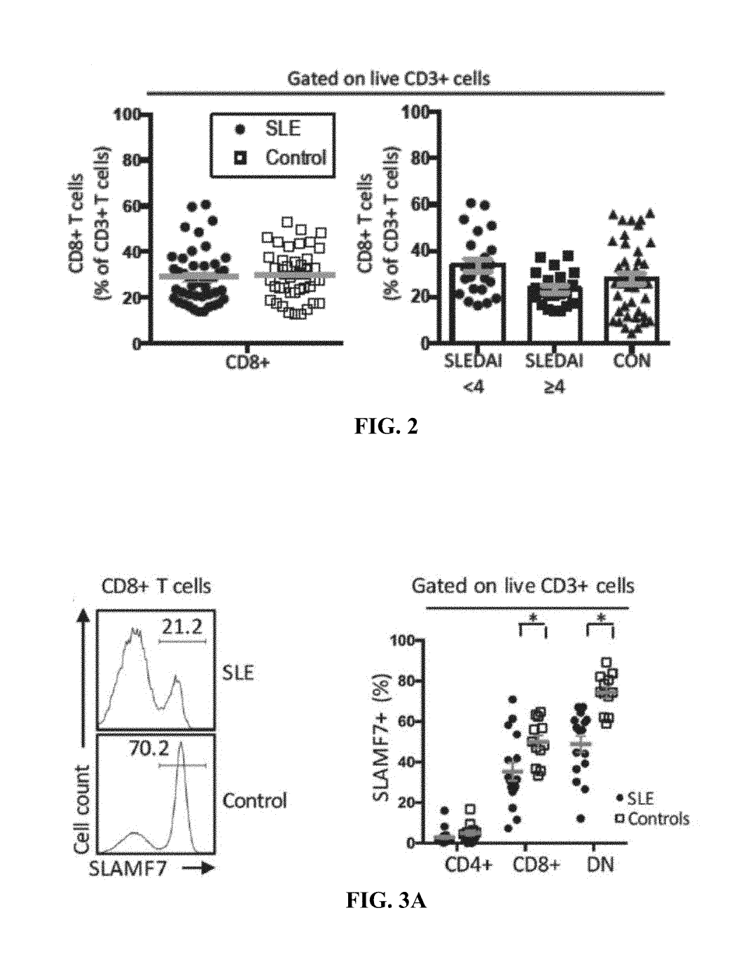

The distribution of CD8+ T cell subsets were screened in the peripheral blood of 45 SLE patients with varying disease activity and 41 healthy controls by assessing cell surface expression of CCR7 and CD45RA. The screening allowed for four differentiated CD8+ T cells subsets to be distinguished, i.e. naïve (CCR7+ CD45RA+), central memory (CM, CCR7+ CD45RA−), effector memory (EM, CCR7−CD45RA−) and terminally differentiated effector memory (TDEM, CCR7−CD45RA+) (FIG. 1A) [23]. Frequency of EM CD8+ T cells was reduced in SLE compared to healthy controls, while cells expressing markers of naïve CD8+ T cells were enriched (FIG. 1B). Moreover, skewed distribution of CD8+ T cells correlated with disease activity, because patients with active disease (as defined by SLEDAI≥4) displayed a statistically significant decrease of EM CD8+ T cells and increase of naïve CD8+ T cells (FIGS. 1C and 1E) compared to patients with inactive disease (SLEDAI<4). CM CD8+ T cells were also decreased in SLE patients but to a lesser degree (FIG. 1D). Of note, there was no difference in the percentage of total CD8+ T cells between SLE patients and controls (FIG. 2).

### Example 2: Decreased SLAMF7 Expression in CD8+ T Cells from SLE Patients

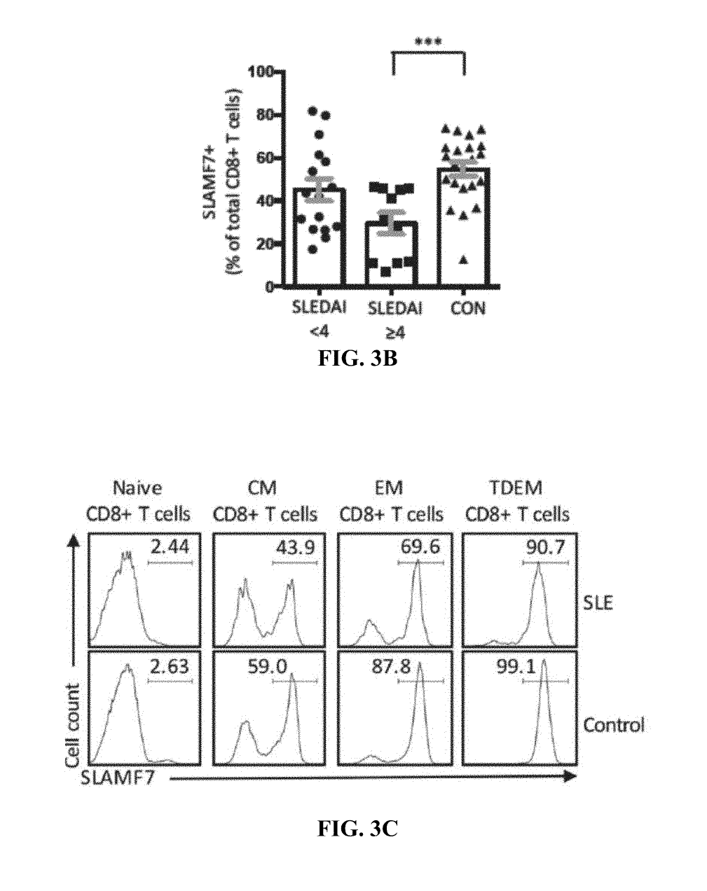

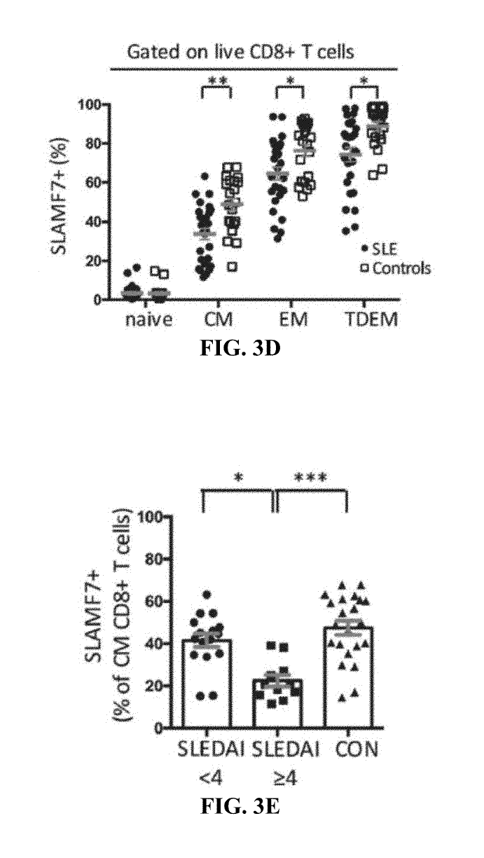

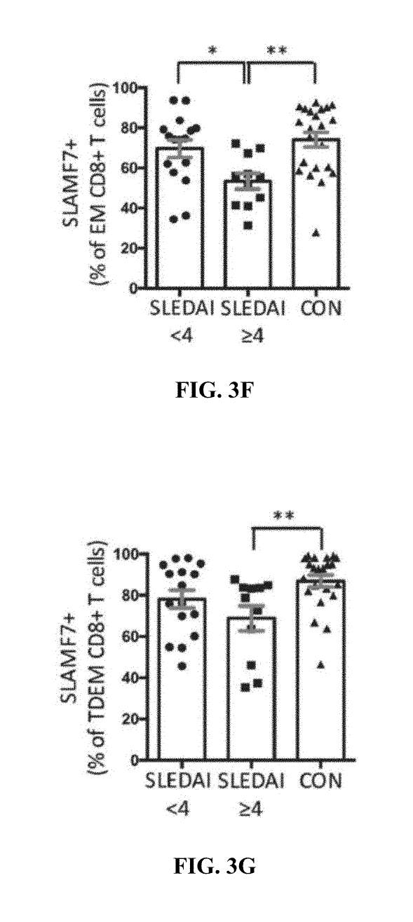

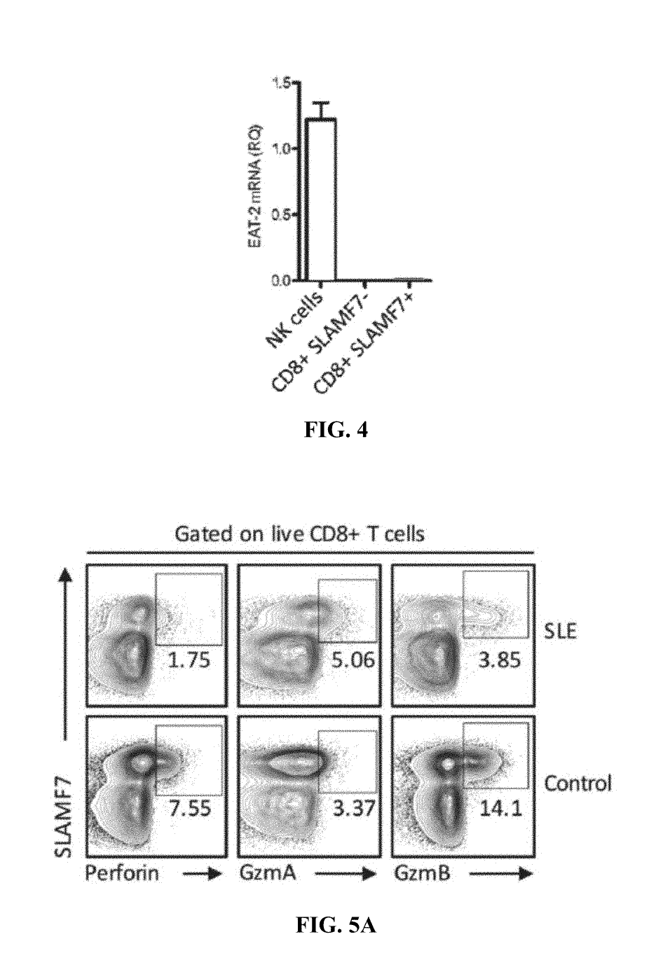

Expression of SLAMF7 was examined in T cells isolated from SLE (n=16 to 27) patients and healthy controls (n=13 to 22). SLAMF7 is mostly expressed by CD8+ T cells, as well as double negative (DN) T cells (FIG. 3A), a T cell subset that expresses CD3 but has lost CD4 and CD8 expression. In contrast, expression of SLAMF7 on CD4+ T cells is very low. Expression of SLAMF7 was found reduced in CD8+ T and DN cells isolated from SLE patients compared to healthy subjects (FIG. 3A). Reduced SLAMF7 expression correlated with disease activity because SLE patients with active disease display less SLAMF7 expression than inactive patients (FIG. 3B). Because the distribution of CD8+ T cell subsets is altered in SLE patients (FIG. 1), the expression of SLAMF7 on each CD8+ T cells differentiated subsets (i.e., naïve, CM, EM, TDEM) was examined. It was observed that SLAMF7 expression increases over cell differentiation: its expression is low in naïve CD8+ T cells, whereas most EM and TDEM express SLAMF7 (FIGS. 3C and 3D). Importantly, each of the CD8+ T cells differentiated subset from SLE patients displayed decreased amounts of SLAMF7 compared to controls (FIGS. 3C and 3D). Decreased SLAMF7 expression was prominent among CD8+ T cells isolated from SLE patients with active disease compared to those with inactive disease (FIGS. 3E-3G). Because it has been previously reported that SLAMF7 binds to EAT-2 in NK cells to transmit downstream signaling, the expression of EAT-2 in CD8+ SLAMF7− and CD8+ SLAMF7+ T cells by qPCR was assessed. EAT-2 gene expression in CD8+ T cells was not detected (FIG. 4).

In summary, SLAMF7 expression increases over CD8+ T cells differentiation and is mostly expressed by EM and TDEM CD8+ T cells. Moreover, SLAMF7 expression is decreased in SLE CD8+ T cells compared to healthy controls.

### Example 3: Expression of SLAMF7 by Effector CD8+ Cytotoxic T Cells

To further examine the properties of SLAMF7+ CD8+ T cells, the co-expression of SLAMF7 and cytolytic enzymes (perforin, GzmA and GzmB) was assessed in CD8+ T cells isolated from peripheral blood of SLE patients (n=18) and controls (n=15). Expression of cytolytic enzyme perforin, GzmA and GzmB was found to be restricted to SLAMF7+ CD8+ T cells (FIGS. 5A and 5B). As previously described [7], an altered expression of cytolytic enzymes was found in SLE patients in comparison to healthy controls: perforin and GzmB expression are reduced, while GzmA expression is slightly increased.

In summary, SLAMF7 expression defines effector cytotoxic CD8+ T cells and its expression is decreased in SLE and is associated with an altered expression of CD8+ T cell cytotoxic enzymes.

### Example 4: SLAMF7 Engagement Restores the Antiviral CD8+ T Cell Response that is Impaired in SLE Patients

To examine the degranulation capacity of CD8+ T cells in response to viral antigens, PBMC isolated from SLE patients (n=8) or healthy individuals (n=8) were stimulated with a mix of MHC class I restricted T cell epitopes from human CMV, EBV and influenza virus (CEF) [7, 24, 25]. PBMC were stimulated with or without anti-SLAMF7 mAb. Cells were stained for CD3, CD8, CD107a (Lysosomal-associated membrane protein 1 (LAMP-1), a marker of degranulation) and IFNγ, and the proportion CD8+ T cells that were CD107a/IFNγ double positive, a population that has been associated with anti-viral protective immunity [26, 27]. Staphylococcal enterotoxin B (SEB) served as a positive control, were examined.

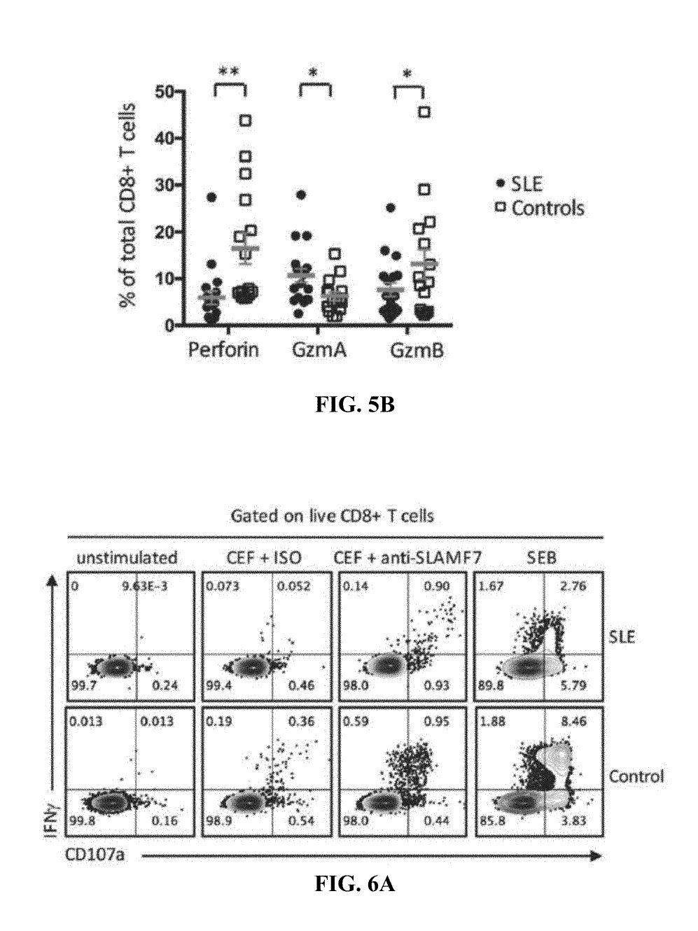

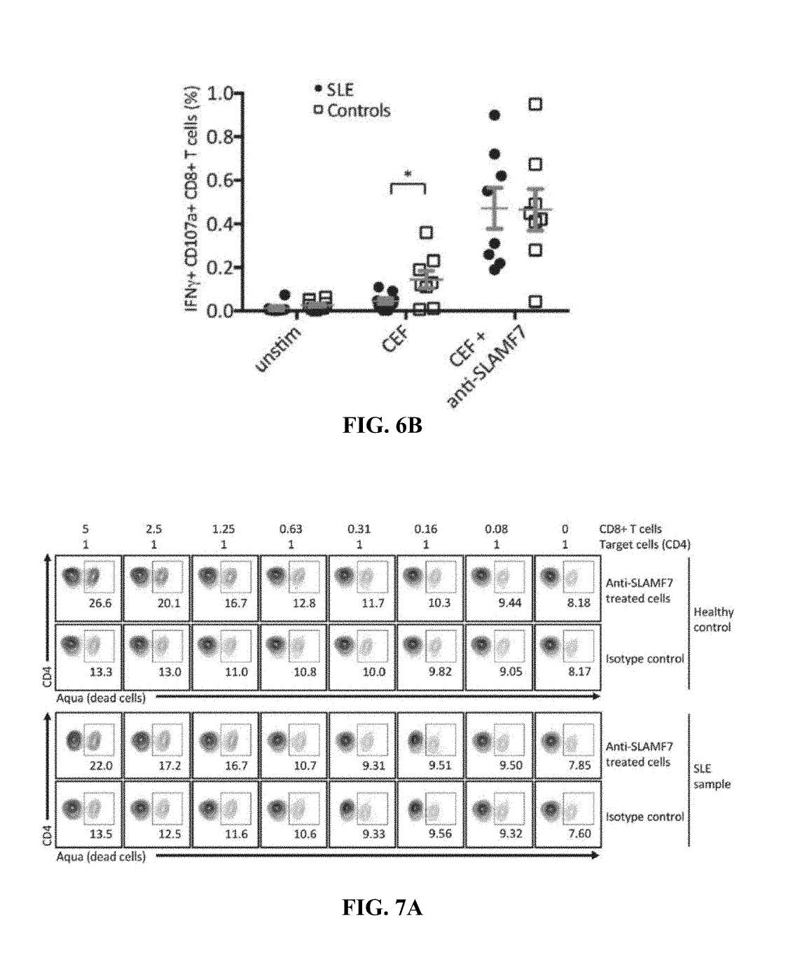

SLAMF7 engagement with a specific mAb increased CD8+ T cells response to CEF stimulation by enhancing the frequency of CD8+CD107a/IFNγ double positive T cells (FIGS. 6A and 6B). Compared to healthy control, SLE patients have fewer CD8+CD107a/IFNγ double positive T cells in response to CEF (FIGS. 6A and 6B). In the presence of anti-SLAMF7 mAb, the frequency of SLE CD8+CD107a/IFNγ double positive T cells in response to CEF stimulation was restored to levels comparable to the one observed in healthy control (FIGS. 6A and 6B).

### Example 5: SLAMF7 Engagement Enhances the Cytolytic Activity of CD8+ T Cells in Response to Viral Antigens

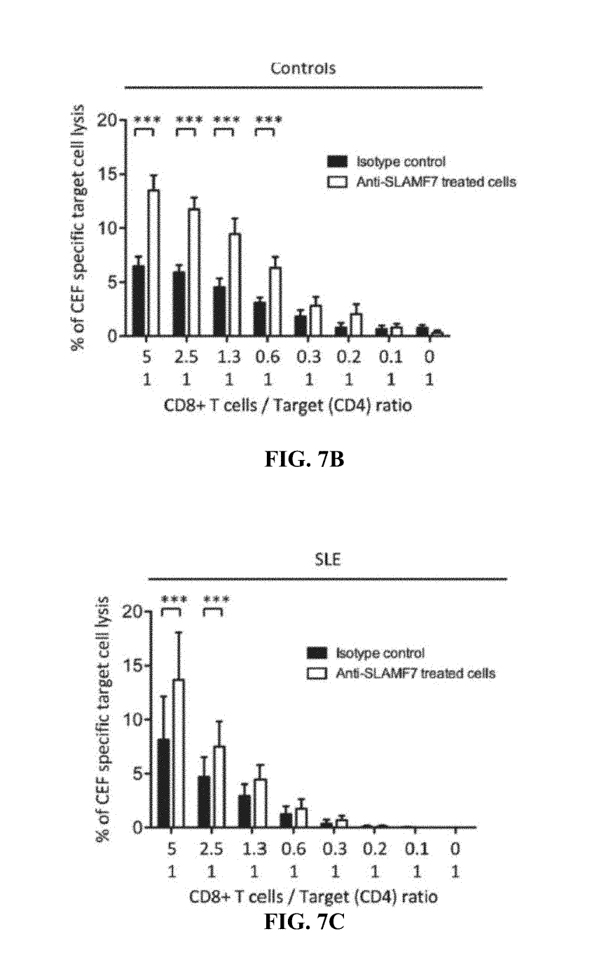

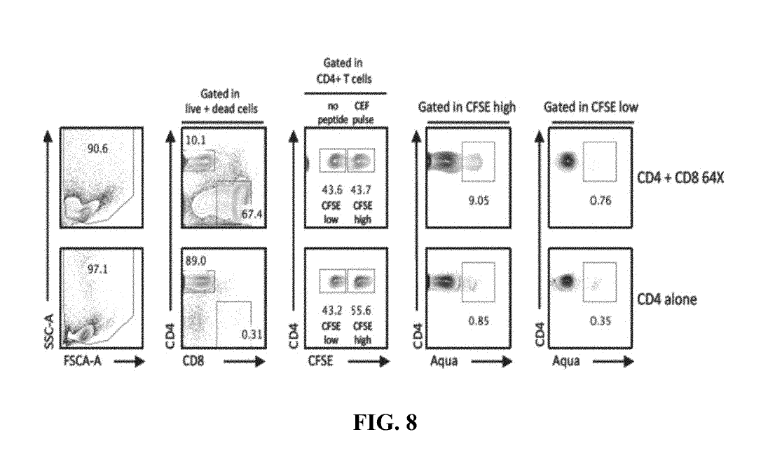

Because SLAMF7 engagement favors CD8+ T cell degranulation upon viral antigenic stimulation, it was investigated whether SLAMF7 activation can trigger CD8+T cell-mediated lysis of target cells. A flow cytometry-based assay was performed to measure the capacity of CEF-multi-specific CD8+ T cells to kill target autologous CD4+ T cells loaded with cognate CEF peptide (modified from reference [28]). Non-loaded autologous CD4+ T cells were used as a background control for target cell death. Expanded effector CD8+ T cells were co-cultured at different ratios with target cells. CEF loaded target CD4+ T cell lysis was augmented in the presence of increasing numbers of CEF antigen multi-specific effector CD8+ T cells (FIGS. 7A-7C and 8).

When CEF expanded CD8+ T cells were activated with anti-SLAMF7 mAb prior to co-culture with target CD4+ T cells, killing of CEF loaded autologous target CD4+ T cells was significantly increased compared to cells treated with an isotype control antibody (FIGS. 7A-7C). In samples from SLE patients, treatment with anti-SLAMF7 mAb, compared to treatment with an isotype control, also enhanced effector CD8+ T cell lysis of target cells in response to CEF peptide (FIG. 7C). SLAMF7-mediated enhancement of target cell lysis in response to CEF peptides was similar in SLE patients and controls (FIGS. 7B and 7C).

In summary, SLAMF7 engagement enhanced CD8+ T cytotoxicity in response to viral antigenic peptides.

### REFERENCES

1. Tsokos, G. C., Systemic lupus erythematosus. N Engl J Med, 2011. 365(22): p. 2110-21.
2. Comte, D., M. P. Karampetsou, and G. C. Tsokos, T cells as a therapeutic target in SLE. Lupus, 2015. 24(4-5): p. 351-63.
3. Stohl, W., Impaired polyclonal T cell cytolytic activity. A possible risk factor for systemic lupus erythematosus. Arthritis Rheum, 1995. 38(4): p. 506-16.
4. Cohen, P. L. and R. A. Eisenberg, Lpr and gld single gene models of systemic autoimmunity and lymphoproliferative disease. Annu Rev Immunol, 1991. 9: p. 243-69.
5. Peng, S. L., et al., Perforin protects against autoimmunity in lupus-prone mice. J Immunol, 1998. 160(2): p. 652-60.
6. Tsokos, G. C., I. T. Magrath, and J. E. Balow, Epstein-Barr virus induces normal B cell responses but defective suppressor T cell responses in patients with systemic lupus erythematosus. J Immunol, 1983. 131(4): p. 1797-801.
7. Kis-Toth, K., et al., Selective Loss of Signaling Lymphocytic Activation Molecule Family Member 4-Positive CD8+ T Cells Contributes to the Decreased Cytotoxic Cell Activity in Systemic Lupus Erythematosus. Arthritis Rheumatol, 2016. 68(1): p. 164-73.
8. Puliaeva, I., R. Puliaev, and C. S. Via, Therapeutic potential of CD8+ cytotoxic T lymphocytes in SLE. Autoimmun Rev, 2009. 8(3): p. 219-23.
9. Cannons, J. L., S. G. Tangye, and P. L. Schwartzberg, SLAM family receptors and SAP adaptors in immunity. Annu Rev Immunol, 2011. 29: p. 665-705.
10. Romero, X., J. Sintes, and P. Engel, Role of SLAM family receptors and specific adapter SAP in innate-like lymphocytes. Crit Rev Immunol, 2014. 34(4): p. 263-99.
11. Wu, N. and A. Veillette, SLAM family receptors in normal immunity and immune pathologies. Curr Opin Immunol, 2016. 38: p. 45-51.
12. Lonial, S., et al., Elotuzumab Therapy for Relapsed or Refractory Multiple Myeloma. N Engl J Med, 2015. 373(7): p. 621-31.
13. Hsi, E. D., et al., CS1, a potential new therapeutic antibody target for the treatment of multiple myeloma. Clin Cancer Res, 2008. 14(9): p. 2775-84.
14. Collins, S. M. et al., Elotuzumab directly enhances NK cell cytotoxicity against myeloma via CS1 ligation: evidence for augmented NK cell unction complementing ADCC. Cancer Immunol. Immunother. 2013:62:1841-9.
15. Detre, C., et al., SLAM family receptors and the SLAM-associated protein (SAP) modulate T cell functions. Semin Immunopathol, 2010. 32(2): p. 157-71.
16. Karampetsou, M. P., et al., Decreased SAP Expression in T Cells from Patients with Systemic Lupus Erythematosus Contributes to Early Signaling Abnormalities and Reduced L-2 Production. J Immunol, 2016. 196(12): p. 4915-24.
17. Comte et al., Engagement of SLAMF3 enhances CD4+ T cell sensitivity to IL-2 and favors regulatory T-cell polarization in systemic lupus erythematosus. PNAS USA 2016:113:9321-6.
18. Hagberg, N., et al., Systemic lupus erythematosus immune complexes increase the expression of SLAM family members CD319 (CRACC) and CD229 (LY-9) on plasmacytoid dendritic cells and CD319 on CD56(dim) NK cells. J Immunol, 2013. 191(6): p. 2989-98.
19. Kim, J. R., et al., Altered expression of signalling lymphocyte activation molecule (SLAM) family receptors CS1 (CD319) and 2B4 (CD244) in patients with systemic lupus erythematosus. Clin Exp Immunol, 2010. 160(3): p. 348-58.
20. Deng, Y. and B. P. Tsao, Genetic susceptibility to systemic lupus erythematosus in the genomic era. Nat Rev Rheumatol, 2010. 6(12): p. 683-92.
21. Tan, E. M., et al., The 1982 revised criteria for the classification of systemic lupus erythematosus. Arthritis Rheum, 1982. 25(11): p. 1271-7.
22. Bombardier, C., et al., Committee on Prognosis Studies in SLE. Derivation of the SLEDAI: a disease activity index for lupus patients. Arthritis Rheum. 1992:35:630-40.
23. Sallusto, F., et al., Two subsets of memory T lymphocytes with distinct homing potentials and effector functions. Nature, 1999. 401(6754): p. 708-12.
24. Currier, J. R., et al., A panel of MHC class I restricted viral peptides for use as a quality control for vaccine trial ELISPOT assays. J Immunol Methods, 2002. 260(1-2): p. 157-72.
25. Mwau, M, et al., Design and validation of an enzyme-linked immunospot assay for use in clinical trials of candidate HIV vaccines. AIDS Res. Hum.

Retroviruses 2002:18:611-8.
26. Seder, R. A., P. A. Darrah, and M. Roederer, T-cell quality in memory and protection: implications for vaccine design. Nat Rev Immunol, 2008. 8(4): p. 247-58.
27. Cellerai, C., et al., Proliferation capacity and cytotoxic activity are mediated by functionally and phenotypically distinct virus-specific CD8 T cells defined by interleukin-7R{alpha} (CD127) and perforin expression. J Virol, 2010. 84(8): p. 3868-78.
28. Noto, A., P. Ngauv, and L. Trautmann, Cell-based flow cytometry assay to measure cytotoxic activity. J Vis Exp, 2013(82): p. e51105.

SEQUENCES

SEQ ID NO: 1 (DNA; F Primer)
TGTGCCTCTGTGTCTCGTTTA
SEQ ID NO: 2 (DNA: R Primer)
ACCACCATCCCCTGATTTGG
SEQ ID NO: 3 (DNA: F Primer)
AGAGCTACGAGCTGCCTGAC
SEQ ID NO: 4 (DNA: R Primer)
AGCACTGTGTTGGCGTACAG

It is claimed:

1. A method of enhancing an immune response in a subject with systemic lupus erythematosus (SLE), the method comprising administering to the subject an effective amount of an anti-signaling lymphocytic activation molecule family member 7 (SLAMF7)-antibody or antigen binding fragment thereof, wherein administration of an effective amount of the anti-SLAMF7 antibody or antigen binding fragment thereof results in an enhanced immune response in the subject with SLE.

2. The method of claim 1, wherein the anti-SLAMF7 antibody or antigen binding fragment thereof is selected from the group consisting of a synthetic antibody, a multi-specific antibody, a chimeric antibody, a single domain antibody, a heavy chain-only antibody, a polyclonal antibody, and a monoclonal antibody.

3. The method of claim 2, wherein the anti-SLAMF7 antibody or antigen binding fragment thereof is a monoclonal antibody.

4. The method of claim 2, wherein the SLAMF7 antibody or antigen binding fragment thereof is a humanized antibody or a humanized antigen binding fragment thereof.

5. The method of claim 1, wherein enhancing an immune response comprises treating or preventing an infectious disease in the subject with SLE.

6. The method of claim 5, wherein the infectious disease is selected from the group consisting of a bacterial infection, a viral infection, and a fungal infection.

7. The method of claim 1, wherein the anti-SLAMF7 antibody or antigen binding fragment thereof is administered to the subject in a pharmaceutical composition.

8. The method of claim 7, wherein the pharmaceutical composition further comprises an adjuvant.

9. The method of claim 7, wherein the pharmaceutical composition is administered prophylactically or therapeutically.

10. The method of claim 7, wherein the route of administration of the pharmaceutical composition to the subject is selected from the group consisting of intramuscularly, subcutaneously, parenterally, orally, intranasally, intradermally, transdermally, intraperitoneally, intratracheally, topically, rectally, and pulmonarily.

11. A method of enhancing degranulation and cytotoxicity of CD8+ T cells in a subject in need of treatment of systemic lupus erythematosus (SLE), the method comprising administering to the subject an effective amount of an antibody that binds a signaling lymphocytic activation molecule family member 7 (SLAMF7), or an antigen binding fragment thereof, wherein administration of the antibody that binds SLAMF7, or an antigen binding fragment thereof to the subject results in an enhancement of degranulation and cytotoxicity of CD8+ T cells in the subject in need of treatment of SLE.

12. The method of claim 11, wherein the anti-SLAMF7 antibody or antigen binding fragment thereof is selected from the group consisting of a synthetic antibody, a multi-specific antibody, a chimeric antibody, a single domain antibody, a heavy chain-only antibody, a polyclonal antibody, and a monoclonal antibody.

13. The method of claim 12, wherein the anti-SLAMF7 antibody or antigen binding fragment thereof is a monoclonal antibody.

14. The method of claim 11, wherein the SLAMF7 antibody or antigen binding fragment thereof is a humanized antibody or humanized antigen binding fragment thereof.

15. The method of claim 11, wherein the anti-SLAMF7 antibody or antigen binding fragment thereof is administered to the subject in a pharmaceutical composition.

16. The method of claim 15, wherein the pharmaceutical composition further comprises an adjuvant.

17. The method of claim 15, wherein the pharmaceutical composition is administered prophylactically or therapeutically.

18. A method of enhancing an immune response in an immunocompromised subject, the method comprising administering to the immunocompromised subject an effective amount of an anti-signaling lymphocytic activation molecule family member 7 (SLAMF7)-antibody or antigen binding fragment thereof, wherein administration of an effective amount of the anti-SLAMF7 antibody or antigen binding fragment thereof results in an enhanced immune response in the immunocompromised subject.

19. The method of claim 18, wherein enhancing an immune response comprises treating or preventing an infectious disease in the immunocompromised subject.

20. The method of claim 19, wherein the infectious disease is selected from the group consisting of a bacterial infection, a viral infection, and a fungal infection.
